SEQUENCE LISTING (1 sequence)

SEQ ID NO: 1 - protein, 19 aa, Unknown
  Description of Unknown ASARM sequence
  MOD_RES (12)..(12): Ser(PO3H2)
  MOD_RES (14)..(14): Ser(PO3H2)
  MOD_RES (16)..(16): Ser(PO3H2)
RDDSSESSDSGSSSESDGD

(19) United States
(12) Patent Application Publication
(10) Pub. No.: US 2022/0105151 A1
(43) Pub. Date: Apr. 7, 2022

(54) ASARM TREATMENT FOR KIDNEY AND MINERALIZATION DISORDERS AND HYPERPHOSPHATEMIA

(71) Applicant: The University of Kansas, Lawrence, KS (US)

(72) Inventor: Peter S. N. Rowe, Overland Park, KS (US)

(21) Appl. No.: 17/495,556

(22) Filed: Oct. 6, 2021

Related U.S. Application Data
(60) Provisional application No. 63/088,847, filed on Oct. 7, 2020.

(51) Int. Cl. A61K 38/17; A61P 13/12

(52) U.S. Cl. CPC A61K 38/1709; A61P 13/12

(57) ABSTRACT

A method of treating or inhibiting a kidney disorder can include administering a therapeutically effective amount of the ASARM peptide to provide a treatment for the kidney disorder (treat disease) and/or inhibit development of the kidney disorder (prophylactic). The kidney disorder can be selected from the group consisting of chronic kidney disease mineral bone disorder (CKD-MBD), calciphylaxis, nephrogenic systemic fibrosis (NSF), end stage renal disease, and combinations thereof. The therapy can include inhibiting vascular calcification (VC), hard tissue calcification, soft tissue calcification, mineralization, or combinations thereof in the subject with the ASARM peptide. A method of inhibiting mineralization can include administering a therapeutically effective amount of the ASARM peptide to provide a treatment for inhibit mineralization in the subject. A method of treating hyperphosphatemia can include administering a therapeutically effective amount of the ASARM peptide to a subject to provide a treatment for hyperphosphatemia.

### CROSS-REFERENCE TO RELATED APPLICATIONS

This patent application claims priority to U.S. Provisional Application No. 63/088,847 filed Oct. 7, 2020, which provisional is incorporated herein by specific reference in its entirety.

### U.S. GOVERNMENT RIGHTS

This invention was made with government support under DK111693 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created Oct. 6, 221, is named K1262_10087US02_SL.txt and is 776 bytes in size.

### BACKGROUND

### Field

The present disclosure relates to the ASARM peptide, and uses thereof in treatment of kidney disorders, mineralization disorders, and hyperphosphatemia. More particularly, the ASARM can be used in methods of treating chronic kidney disease disorder (CKD-MBD), calciphylaxis, and nephrogenic systemic fibrosis (NSF), and others.

### Description of Related Art

Chronic kidney disease involves a gradual loss of kidney function. The kidneys filter wastes and excess fluids from blood, which are then removed in urine. Advanced chronic kidney disease can cause dangerous levels of fluid, electrolytes and wastes to build up in the body. Treatment for chronic kidney disease focuses on slowing the progression of kidney damage, usually by controlling the cause. However, even controlling the cause may not stop kidney damage from progressing. Chronic kidney disease can progress to end-stage kidney failure, which is fatal without artificial filtering (dialysis) or a kidney transplant.

Chronic kidney disease-mineralization bone disorder (CKD-MBD) is manifest by abnormalities in blood, parathyroid hyperplasia, renal osteodystrophy and mineralized vascular or soft tissue calcification. Abnormalities, such as hyperphosphatemia, hypocalcemia, increased parathyroid hormone (PTH), increased FGF23 and decreased 1,25-dihydroxyvitamin D (1,25 D) occur as CKD-MBD progresses. These changes induce abnormal bone remodeling and extra-skeletal calcification that contribute to mortality and morbidity in affected patients. The Kidney Diseases Improving Global Outcomes (KDIGO) guidelines for treating CKD-MBD recommend reducing non-phytates inorganic phosphate (Pi) levels to the normal range by using Pi binders or dietary modification. Specifically, keeping one, two or optimally all three sera metrics of calcium (Ca), Pi and PTH within normal ranges significantly decreases morbidity and mortality. Notably, hyperphosphatemia often leads to death because of the adverse effects on vascular calcifications. Further, the high phosphate levels play a major role in the abnormal bone disorders, hypocalcemia, secondary hyperparathyroidism and increased FGF23 levels [Elias, R M, et al.; CKD-MBD: from the Pathogenesis to the Identification and Development of Potential Novel Therapeutic Targets. Curr Osteoporos Rep, 16: 693-702, 2018]. Also, increased phosphate is toxic and phenotypically alters vascular smooth muscle cells (VSMC) towards osteoblastic differentiation [Shroff, R, et al.; Mechanistic insights into vascular calcification in CKD. J Am Soc Nephrol, 24: 179-189, 2013; Shroff, R: Phosphate is a vascular toxin. Pediatr Nephrol, 28: 583-593, 2013; Rinat, C, et al.; A comprehensive study of cardiovascular risk factors, cardiac function and vascular disease in children with chronic renal failure. Nephrol Dial Transplant, 25: 785-793, 2010]. The change in phenotype is accompanied by extra-skeletal mineralization, inflammation and cardiovascular disease.

Additionally, calciprotein nanoparticles (CPPs) play a major role in nephrotoxicity and vascular calcification in CKD-MBD. Specifically, a recent study concluded calcium phosphate particles in the renal tubular fluid present an effective therapeutic target to decelerate nephron loss during the course of aging and CKD progression [Shiizaki K, et al., Calcium phosphate microcrystals in the renal tubular fluid accelerate chronic kidney disease progression. J Clin Invest. 2021; Epub Jun. 30, 2021]. As CKD-MBD progresses, circulating concentrations of phosphate increase above a certain threshold and free phosphate binds to calcium to form “calcium-phosphate-nanocrystal” calciprotein particles (CPPs). The nascent CPPs have the capacity to expand in size to form much larger insoluble precipitates. Calciprotein precipitates (CPP) are cytotoxic to the vascular endothelium and induce a phenotypic transformation of vascular smooth muscle cells (VSMC) to osteoblast-like (bone-forming) cells. The CPP induced osteoblast-like cells deposit hydroxyapatite in the arterial wall and are thus an early step in the pathophysiology of vascular calcification in CKD [Shuto E, et al., Dietary phosphorus acutely impairs endothelial function. J Am Soc Nephrol. 2009; 20(7):1504-12, Epub May 2, 2009; Di Marco GS, et al., Increased inorganic phosphate induces human endothelial cell apoptosis in vitro, Am J Physiol Renal Physiol. 2008; 294(6):F1381-7, Epub Apr. 4, 2008; Steitz S A, et al., Smooth muscle cell phenotypic transition associated with calcification: upregulation of Cbfa1 and downregulation of smooth muscle lineage markers. Circ Res. 2001; 89(12):1147-54, Epub Dec. 12, 2001; Jono S, et al., Phosphate regulation of vascular smooth muscle cell calcification. Circ Res. 2000; 87(7):E10-7, Epub Sep. 29, 2000]. Therefore, there is a clear need to develop treatment strategies to treat kidney diseases, and control hyperphosphatemia as well as prevent progression of vascular calcification in CKD-MBD and end stage renal disease (ESRD) patients on dialysis.

### SUMMARY

In some embodiments, a method of treating or inhibiting a kidney disorder is provided. Such a method can include: providing an ASARM peptide; and administering a therapeutically effective amount of the ASARM peptide to a subject to provide a treatment for the kidney disorder in the subject and/or inhibit development of the kidney disorder in the subject. In some aspects, the kidney disorder is selected from the group consisting of chronic kidney disease mineral bone disorder (CKD-MBD), calciphylaxis, nephrogenic systemic fibrosis (NSF), end stage renal disease, and combinations thereof. In some aspects, the method can include inhibiting vascular calcification (VC), hard tissue calcification, soft tissue calcification, mineralization (e.g., atherosclerotic plaque deposition), or combinations thereof in the subject with the ASARM peptide. In some aspects, the method can include treating or inhibiting a metabolism abnormality of at least one of calcium, phosphorus, PTH, or vitamin D metabolism with the ASARM peptide. In some aspects, the method can include treating or inhibiting an abnormality in at least one of bone turnover, mineralization, bone volume, bone growth, or bone strength with the ASARM. In some aspects, the method can include treating or inhibiting hyperphosphatemia with the ASARM. In some aspects, the method can include inhibiting nanocrystal or calciprotein particle formation with the ASARM. In some aspects, the method can include inhibiting vascular smooth muscle cells (VSMC) from transitioning to an osteoprogenitor phenotype cell. In some aspects, the kidney disorder is CKD-MBD. In some aspects, the kidney disorder is calciphylaxis, which is inhibited from formation of lesions. In some aspects, the kidney disorder is NSF. In some aspects, the subject is administered the ASARM after receiving gadolinium binding contrast agents (GBCAs). In some aspects, the ASARM is infused into the subject or otherwise administered in any way.

In some embodiments, a method of inhibiting mineralization in a subject is provided. The method can include administering a therapeutically effective amount of the ASARM peptide to a subject to provide a treatment for inhibiting mineralization in the subject. In some aspects, the ASARM inhibits mineralization of a soft tissue. In some aspects, the soft tissue is selected from brain, heart, renal, liver, muscle, dermal, vessel, tubule, nephron, or combinations thereof. In some aspects, the inhibited mineralization is inhibited calcification. In some aspects, the ASARM binds with mineral crystals.

In some embodiments, a method of treating hyperphosphatemia is provided. The method can include administering a therapeutically effective amount of the ASARM peptide to a subject to provide a treatment for hyperphosphatemia. In some aspects, the ASARM reduces serum phosphate levels in the subject. In some aspects, the ASARM inhibits uptake of phosphate by the digestive tract or kidney of the subject.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates the biological pathways for hyperphosphatemia leading to nanocrystals, which induce vascular smooth muscle cells to transform to osteoprogenitor cells, which in turn transform to osteoblasts that can cause vascular calcification, which can be inhibited with ASARM peptide. The ASARM peptide also has inhibitors.

FIGS. 1A-1L show the measurements for different biomarkers at the start point and end point for ASARM infusion. As shown, ASARM infusion corrects key serum chemistries in sub-total 5/6 Nephrectomy rats (male, 6 weeks, 250 g) fed a high phosphate diet (HPO₄; 2% P, 2000 IU Vitamin D and 0.8% Ca; Teklad #170496). Measurements were taken at the start and end of the experiment (28 days infusion). Index: SHAMX=sham operated, 56NEPHX=sub-total 5/6^{th} Nephrectomy (N=6). Index: BUN=Blood Urea Nitrogen, ALT=Alanine Amino Transferase; AST=Aspartate Amino Transferase; VEH=vehicle; ASARM=ASARM phosphorylated peptide, as provided herein. Two-way ANOVA results not RM: interaction, time and treatment group with percentage of total variation of 19.95, 9.240 and 65.48 respectively with p<0.0001.

FIGS. 2A-2D show measurements for different biomarkers at the start point and end point for ASARM infusion. ASARM infusion corrects key serum chemistries in sub-total 5/6 Nephrectomy rats fed a high phosphate diet (HPO₄; 2% P, 2000 IU Vitamin D and 0.8% Ca; Teklad #170496). Measurements were taken at the start and end of the experiment (28 days infusion). Index: SHAMX=sham operated, 56NEPHX=sub-total 5/6th Nephrectomy (N=6). Two-way ANOVA results not RM: interaction, time and treatment group with percentage of total variation of 19.95, 9.240 and 65.48 respectively with p<0.0001.

FIG. 3A shows μCT scans of rat hearts showing significantly reduced heart calcification in ASARM infused sub-total 5/6th Nephrectomy rats fed a high phosphate diet (HPO₄; 2% P, 2000 IU Vitamin D and 0.8% Ca; Teklad #170496), representative heart μCT scans Index: SHAMX=sham operated, 56-NEPHREX=sub-total 5/6th Nephrectomy (N=6).

FIG. 3B shows graphical output of heart mineral volume over total tissue volume (MV/TV). Index: SHAMX=sham operated, 56NEPHREX=sub-total 5/6th Nephrectomy (N=6). One-way ANOVA with Tukey multiple comparison; *=p<0.0032, ns=not significant—as shown on the graph. Note with an unpaired t-test (two-tailed) the SHAM group and 56NEPHREX ASARM group were significantly different p=0.0296. Also, the 56NEPHREX vehicle versus 56NEPHREX ASARM group remained significantly different with an unpaired t-test (two-tailed) p=0.0036.

FIG. 4A shows representative aorta photos and corresponding μCT scans of vehicle and ASARM peptide treated sub-total 5/6th nephrectomy rats, which shows ASARM infusion prevents medial calcification of the aorta.

FIG. 4B shows the image of aorta histology sections (7 μM) stained with Von Kossa (black color=mineral), where extensive medial calcification is prevented in rats infused with ASARM peptide.

FIG. 4C includes a graph showing aorta mineral volume/total tissue volume (MV/TV) calculated using μCT analysis is reduced in rats infused with ASARM. Index: SHAMX=sham operated, 56-NEPHREX=sub-total 5/6th Nephrectomy (N=6). Unpaired t-test (two-tailed) the 56NEPHREX vehicle versus 56NEPHREX ASARM were significantly different p=0.0238. No detectable mineral deposition occurred with the SHAM treated rats (not shown). Rats were fed the high phosphate diet (HPO₄; 2% P, 2000 IU Vitamin D and 0.8% Ca; Teklad #170496).

FIG. 5A includes an image of representative μCT scans of SHAM operated and 5/6 nephrectomy rats, which shows the ASARM peptide infusion decreases renal mineralization. Note significant decreased calcification in ASARM treated 56 NEPHREX rats.

FIG. 5B includes a graph that shows the graphical presentation of μCT quantification of renal total mineral volume versus total volume ratio (MV/TV). Index: SHAMX=sham operated, 56-NEPHREX=sub-total 5/6th Nephrectomy (N=6). Results for the one-way ANOVA with Tukey multiple comparison test were: 56NEPHREX Vehicle versus 56NEPHREX ASARM p<0.0001 and 56NEPHREX Vehicle versus SHAM p<0.0003. Statistics shown on the graph represent unpaired t-test (two-tailed). No significant difference occurred between 56NEPHREX ASARM v SHAM groups for both tests.

FIG. 5C includes an image of renal histology section stained with Von Kossa showing mineral deposition (black color) in 7 μM section of 56NEPHREX rat vehicle treated kidney (×20).

FIG. 6A shows representative micro computed tomography (μCT) scans of femur diaphysis, which shows that ASARM Significantly improves bone volume/total volume (BV/TV) in 5/6 Nephrectomy rats fed a high phosphate diet.

FIG. 6B shows the femur diaphysis bone volume images.

FIG. 6C includes a corresponding graph showing bone volume/total tissue volume (BV/TV) calculated using μCT analysis. Note, BV/TV was corrected in 56NEPHREX rats infused with ASARM peptide. Index: SHAMX=sham operated, 56NEPHREX=sub-total 5/6th Nephrectomy (N=6). Results for the one-way ANOVA with Tukey multiple comparison test were: 56NEPHREX Vehicle versus 56NEPHREX ASARM p=0.0358, and SHAM versus 56NEPHREX vehicle p=0.0133. Unpaired t-test (two-tailed) for the 56NEPHREX vehicle versus 56NEPHREX ASARM were also significantly different p=0.0329. No significant difference between SHAM versus 56NEPHREX ASARM groups were measured for both tests. Rats were fed the high phosphate diet (HPO₄; 2% P, 2000 IU Vitamin D and 0.8% Ca; Teklad #170496).

FIG. 7A shows different panels for skin sub-dermal vascular calcification, which is significantly reduced in 5/6th nephrectomy rats infused with ASARM—calciphylaxis-like lesions are absent: (Panel A) μCT skin scan showing subdermal calcified blood vessels; (Panel B) Corresponding photograph showing “under skin” surface and blood vessels scanned in (Panel A); (Panel C) Photo showing upper skin surface with “peu' d'orange lesions” that resemble calciphylaxis lesions; (Panel D) Cross sectional μCT x-ray “dicom” image showing evidence of medial vascular calcifications; (Panel E) Corresponding histology—skin cross section (7 μm) stained for mineral with Von Kossa (black color=mineral) ×20; and (Panel E) magnified view at ×40.

FIG. 7B shows a graphical presentation of μCT quantification of skin total mineral volume v total volume ratio (MV/TV). Index: SHAMX=sham operated, 56 NEPHREX=sub-total 5/6th Nephrectomy (N=6). Results for the one-way ANOVA with Tukey multiple comparison test were: 56 NEPHREX Vehicle versus 56 NEPHREX ASARM p=0.022, and SHAM versus 56 NEPHREX vehicle p=0.04. Unpaired t-test (two-tailed) for the 56 NEPHREX vehicle versus 56 NEPHREX ASARM were also significantly different p=0.018. No significant difference between SHAM v 56 NEPHREX ASARM groups were measured for both tests. Rats were fed the high phosphate diet (HPO4; 2% P, 2000 IU Vitamin D and 0.8% Ca; Teklad #170496).

FIG. 8A shows images for the decreased serum phosphate and gut jejunum Na+ dependent phosphate co-transporter NPT2B (Slc34A2) occurs with 56NEPHREX rats infused with ASARM peptide compared to rats infused with vehicle, which shows a representative cross sectional H&E stain of rat jejunum and vehicle treated rats with pictures showing DAB stain (NPT2B (typically brown), phalloidin fluorescence stain for F-actin (green), fluorescence stain for NPT2B (typically red) and merged F-actin and NPT2B. Overlap between F-actin and NPT2B typically appears yellow).

FIG. 8B includes a graph showing NPT2B positive quantification for DAB histology sections (DAB area stain/total tissue area). A significant decrease in NPT2B staining occurs with ASARM treated 56NEPHREX rats. Unpaired t-test (two-tailed) for the 56NEPHREX vehicle v 56NEPHREX ASARM confirmed a significant difference p=0.0218.

FIGS. 9A-9B include graphs that show decreased serum ASARM peptides occur in mice and patients with CKD-MBD. FIG. 9A shows serum ASARM in 16-wk male Alport mice (Col4a^{-/-}) with n=5 per group. FIG. 9B shows serum ASARM in CKD-MBD patients on dialysis with end stage renal disease (ESRD). All ESRD patients had high FGF23 (n=15)—males 65±12 yr. Control males (n=7) 58±15 yr. Unpaired t-test (two-tailed) confirmed a significant differences between control and experimental groups (see graph).

FIG. 10A shows decreased brain calcification occurs in 56NEPHREX rats treated with ASARM peptide. ASARM peptide infusion prevents brain calcification, which includes a sagittal view of a 3D μCT scan of a 56NEPHREX (56NEPHX) vehicle treated rat brain (note calcification deposits), and a coronal section showing location of calcified nidi.

FIG. 10B includes a graph that shows mineral volume/brain tissue volume (MV/TV). ASARM treated rats show significant inhibition of brain calcification. Index: NS=not significant; *=p<0.01 1 way anova and Tukey multiple comparison.

FIG. 11A shows decreased brain calcification occurs and improved vasculature occurs with 56NEPHREX rats treated with ASARM peptide. ASARM peptide prevents brain calcification and vascular abnormalities occur with 56NEPHREX rats, where the image shows the transverse photographic view of representative rat brain.

FIG. 11B shows a sagittal view of a 3D μCT scan of a 56NEPHREX vehicle treated rat brain (note calcification deposits) and a corresponding horizontal plane view of rat brain vasculature scanned by μCT following MicroFil perfusion.

FIG. 12 includes images that show ASARM peptide infusion completely cures calciphylactic skin lesions in 56NEPHREX rats and also severe sub-dermal calcification in rats gadolinium contrast agent (Omniscan™) induced nephrogenic systemic fibrosis (NSF). Skin sections from high phosphate diet rats. Upper row (A) shows μCT representative scanned skin images and the middle row (B) and lower row (C) show corresponding upper and lower skin photographs of the scanned μCT samples shown in row A. Note, ASARM infusion of Omniscan™ treated rats completely prevents the massive dermal calcification. Also, ASARM treatment prevented “calciphylactic-like” lesions in 56NEPHREX rats on high phosphate diet.

FIG. 13A shows the relativeness of the skin and cross section, showing ASARM peptide treatment cures the severe skin calciphylactic pathology and suppresses the macrophage response. Massive sub dermal calcification, fibrosis and macrophage recruitment occurs in 56NEPHREX rats exposed to Omniscan™ and fed the high phosphate diet ((HPO₄). ASARM infusion prevents both pathologies: μCT scan of a representative skin section a 56NEPHREX rat treated with gadolinium contrast agent Omniscan™; Cross sectional view; and Von Kossa histology staining of sample (typically mineral deposition dark brown color).

FIG. 13B shows the ASARM with Omniscan™ and Vehicle with Omniscan™. Immunohistochemistry skin sample of 56NEPHREX rat treated with omniscan™ and infused with ASARM and stained for CD68 macrophage marker, and with 56NEPHREX rat infused with vehicle instead of ASARM (note in the absence of ASARM treatment massive fibrosis and macrophage recruitment reminiscent of NSF.

FIG. 13C includes graphs that show: Von Kossa stain quantitation of skin calcification; Inductively Coupled Mass Spectrometry/High Performance Liquid Chromatography (ICPMS-HPLC) Gadolinium analysis (PPB=parts per billion); and Immunohistochemistry (IHC) quantitation of macrophage maker in representative dermal samples.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology is related to the use of ASARM peptide for the treatment of kidney diseases. ASARM is a small, phosphorylated peptide that can now be used for the treatment of kidney diseases, such as chronic kidney disease mineralization disorder (CKD-MBD), calciphylaxis, and nephrogenic systemic fibrosis (NSF), and the symptoms thereof as well as any mineral deposition disorder, such as vascular calcification. ASARM can be administered as a treatment or prophylaxis for the kidney diseases.

Abnormalities in calcium, phosphorus, PTH, vitamin D metabolism, bone and vascular calcification occur in CKD-MBD. Calciphylaxis involving painful, ulcerative skin lesions is also a major problem associated with CKD-MBD. In NSF, the skin lesions are severe with massive, painful, “fibrosing” ulcerative indurations and red bumps. Now, it has been found that bone ASARM peptides are strong inhibitors of mineralization and induce hypophosphatemia (e.g., treat hyperphosphatemia) by inhibiting phosphate uptake from the gut. The ASARM peptide can now be used for a of CKD-MBD, where ASARM peptides can reverse hyperphosphatemia, reduce soft tissue calcification, correct osteodystrophy, prevent calciphylaxis and improve mortality.

In some embodiments, the ASARM peptide can be used in the prophylaxis or treatment of CKD-MBD. CKD-MBD is a systemic disorder of mineral and bone metabolism due to chronic kidney disease CKD manifest by either one or a combination of the following: 1. Abnormalities of calcium, phosphorus, PTH, or vitamin D metabolism; 2. Abnormalities in bone turnover, mineralization, volume, linear growth, or strength (renal osteodystrophy); and 3. Vascular or other soft tissue calcification. As such, ASARM can be used for treatment of the foregoing conditions associated with CKD-MBD. A rat model of CKD-MBD that has a “5/6th Nephrectomy” (referred to herein as 56NEPHREX, or 56NEPHX) can be used to show that administration (e.g., continuous osmotic pump infusion) of ASARM over a period of 4 weeks corrects hyperphosphatemia, cardiac calcification, vascular calcification, renal calcification, brain calcification, bone abnormalities (renal osteodystrophy) and calciphylaxis, as well as improvement in survival. The data provided herein supports the use of ASARM as a prophylactic and treatment for CKD-MBD patients or any end stage renal disease patients on dialysis as well as for vascular calcification. The data indicates that ASARM can be administered intravenously or orally in order to prophylactically inhibit adverse mineralization by direct action and by inhibiting phosphate uptake from the gut and kidneys by inhibiting sodium dependent phosphate cotransporters (Npt2b, NaPi-IIb, SLC34A2) and (Npt2a, NaPi-IIa, SLC34A1), respectively. Also, administration of ASARM by subdermal injection or topical administration can be used to treat and reverse the serious complication of skin calciphylaxis.

In some embodiments, the ASARM peptide can be used as a prophylactic or treatment of NSF. In some instances, high contrast magnetic resonance imaging (MRI) uses gadolinium binding contrast agents (GBCAs). Subsets of chronic kidney disease (CKD) patients exposed to GBCAs develop NSF, which is a progressive disease that leads to acute morbidity and death. Toxic gadolinium (Gd3+) release from GBCAs is thought to play a role. It is thought that ASARM can induce release of Gd3+, which is coupled to a secondary process that results in neutralization and clearance of gadolinium. Specifically, ASARM may facilitate a “targeted” transfer of Gd3+ to a secondary molecule (possibly by transmetallation) and thereby suppress Gd3+ toxicity and increase clearance. The rat 5/6 Nephrectomy CKD disease model was used and sham operated rats were used as controls (SHAM). Male rats (16 week, 250 gm) were fed a high phosphate diet (2% P, 200 IU Vit D and 0.8% Ca; TEKLAD 170496). ASARM peptide was infused continuously for 4 weeks using subcutaneous implantation of Alzet osmotic pumps. As controls, co-implanted osmotic pumps were used to co-infuse SPR4 peptide, which is a peptide that neutralizes ASARM peptide. Sera collections were taken at the beginning and end of the study. Three consecutive, daily bolus injections of Gd3+-containing contrast agent (Omniscan™, gadodiamide) were given 3 days after pump implantation through surgically implanted jugular-vascular-catheters. NEPHREX rats treated with Omniscan™ and ASARM-peptide developed severe skin pathology, behavioral abnormalities, and joint abnormalities that were consistent with NSF. Computed tomography (μCT) showed renal, brain, heart, and dermal metastatic calcifications and bone defects in Omniscan™ treated Rats. It was found that ASARM peptide treatment corrected the Omniscan™ induced skin, bone and soft tissue mineral abnormalities and corrected the hyperphosphatemia. The study shows that CKD rats fed a high phosphate diet and treated with Omniscan™ develop severe NSF like pathology. It was also shown that ASARM infusion prevents GBCA induced subdermal calcification, which corrects mineral defects and hyperphosphatemia. Thus, one treatment includes ASARM peptides inducing release of free Gd3+ from GBCAs, and also reducing mineralization pathology. In some aspects, the ASARM peptide can be used to inhibit or reduce formations of fibrosis.

In some embodiments, the ASARM peptide can be used in a prophylactic or treatment of calciphylaxis. Calciphylaxis includes painful, ulcerative skin lesions is also a major problem associated with CKD-MBD. It has now been shown that ASARM-peptide infusion prevented the genesis of sub-dermal medial blood vessel calcification and calciphylaxis like lesions in 56NEPHREX rats compared to 56NEPHREX rats infused with vehicle. Thus, ASARM can be used as a prophylactic to prevent or otherwise inhibit onset or development of calciphylaxis, such as in CKD-MBD patients.

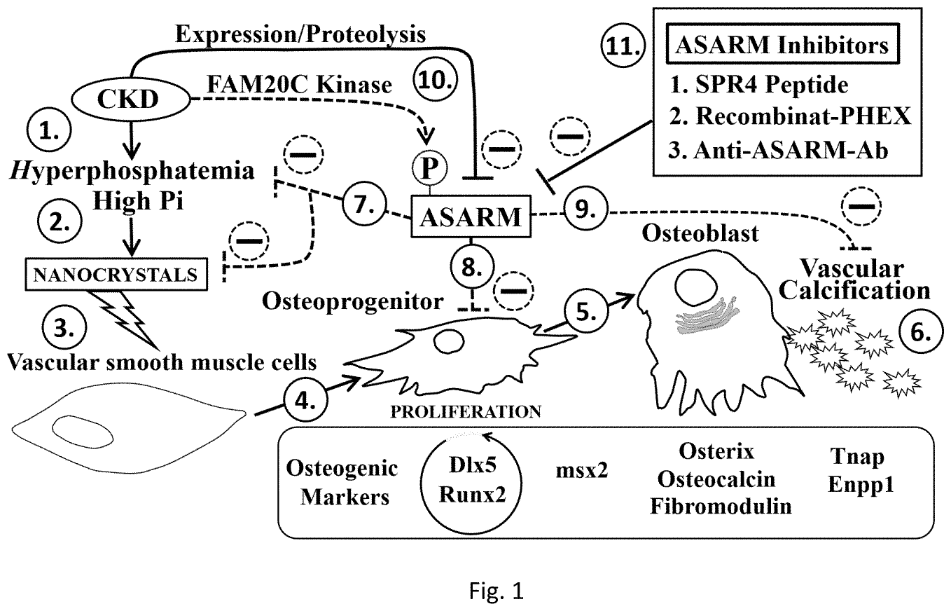

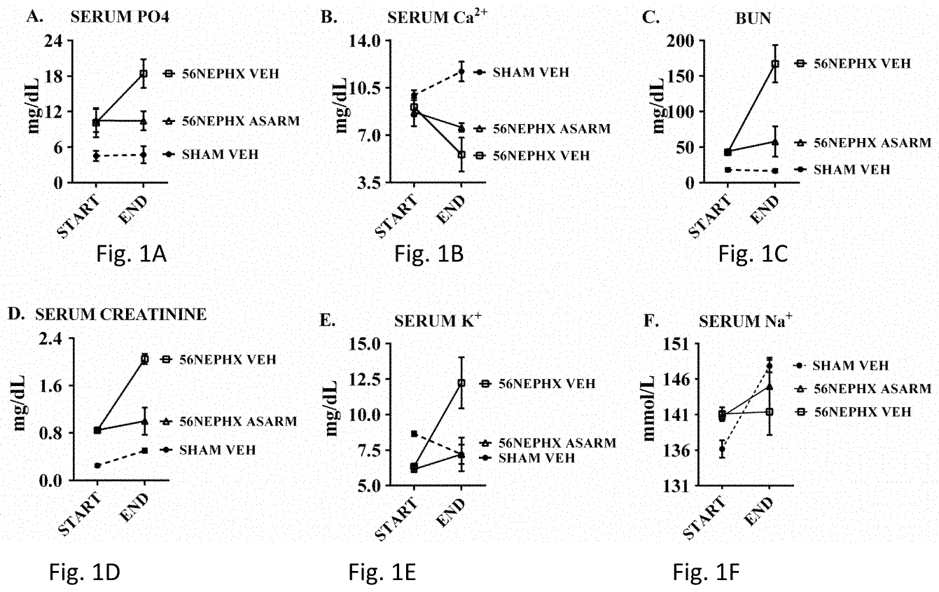

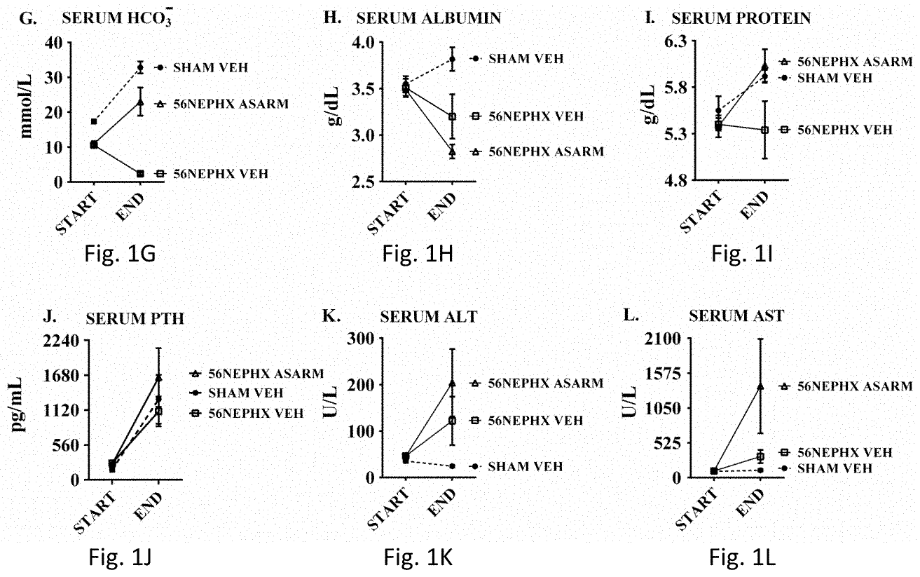

The ASARM peptide has been shown to prevent renal disease that is induced by vascular calcification (VC). The data provided herein indicates that VC is inhibited by the ASARM being multifunctional in: inhibiting osteogenesis, preventing hyperphosphatemia, binding to nanocrystals or calciprotein particles (CPPs) and binding to nascent minerals. FIG. 1 shows a diagram of how ASARM can inhibit VC. The numbers in FIG. 1 correspond to the numbered descriptions provided below: 1). ASARM can inhibit progressive hyperphosphatemia, which is a key feature of chronic kidney disease (CKD); 2). Hyperphosphatemia predisposes a subject to nanocrystal or calciprotein particle (CPP) formation, which can be inhibited by ASARM; 3). CPPs and high Pi both interact with vascular smooth muscle cells (VSMC) and switch on genes that activate phenotypic signal-transition pathways, which can be inhibited by ASARM; 4). VSMC's proliferate and transition to an osteoprogenitor phenotype—key osteogenic markers are activated (Dlx5, Runx2 etc.); 5). Osteogenic transition continues with the genesis of matrix vesicles and the appearance of microcrystalline nidi of hydroxyapatite, which can be inhibited by ASARM; 6). Vascular calcification proceeds with intimal and medial mineral deposition and growth, which can be inhibited by ASARM. Accordingly, FIG. 1 shows the multipronged functionalities of the ASARM peptide that inhibits the overall formation of VC. FIG. 1 shows: 7). ASARM peptides are anti hyperphosphatemic that inhibit renal and intestinal Pi uptake and bind to CPPs; 8) ASARM peptides inhibit osteogenesis and VSMC phenotypic transition; 9). ASARM peptides bind to and prevent the growth of hydroxyapatite and mineral; 10). ASARM administration can overcome problems associated with low ASARM, such as in CKD where abnormal expression, proteolytic processing, and FAM20C kinase phosphorylation results in reduced levels of active bone derived ASARM peptides. Of relevance, ASARM peptide is the only known physiological substrate for PHEX [(Critical reviews in eukaryotic gene expression. 2012; 22(1):61-86. Epub Feb. 22, 2012; Rowe P S, et al., Regulation of Bone-Renal Mineral and Energy Metabolism: The PHEX, FGF23, DMP1, MEPE ASARM Pathway). (Bone Miner Res. 2010; 25(4):695-705. Epub Sep. 25, 2009; Addison W, et al., Phosphorylation-Dependent Inhibition of Mineralization by Osteopontin ASARM Peptides is Regulated by PHEX Cleavage). (J Bone Miner Res. 2008; 23(10):1638-49. Epub Jun. 30, 2008; Addison W, et al., MEPE-ASARM Peptides Control Extracellular Matrix Mineralization by Binding to Hydroxyapatite—An Inhibition Regulated by PHEX Cleavage of ASARM). (Biochem J. 2003; 373(1):271-9; Campos M., Human recombinant endopeptidase PHEX has a strict S1′ specificity for acidic residues and cleaves peptides derived from fibroblast growth factor-23 and matrix extracellular phosphoglycoprotein). (Endocrinology. 2008; 149(4):1757-72. Epub 2007 Dec. 27; Martin A., et al. Degradation of MEPE, DMP1, and release of SIBLING ASARM-peptides (minhibins): ASARM-peptide(s) are directly responsible for defective mineralization in HYP). (Bone. 2005; 36(1):33-46; Rowe P S N., Surface Plasmon Resonance (SPR) confirms MEPE binds to PHEX via the MEPE-ASARM-motif: A model for impaired mineralization in X-linked rickets (HYP)). (Am J Physiol Renal Physiol. 2011; 300(3):F783-91. Epub Dec. 24, 2010; David V., ASARM peptides: PHEX-dependent & independent regulation of serum phosphate). (Bone. 2006; 39(4):773-86; Rowe P S., Correction of the mineralization defect in hyp mice treated with protease inhibitors CA074 and pepstatin)]. Also, PHEX is highly expressed in CKD-MBD [Kidney Int. 2017; 91(6):1436-46, Epub Mar. 21, 2017; Graciolli, F G, et al., The complexity of chronic kidney disease-mineral and bone disorder across stages of chronic kidney disease]. The reduced levels of ASARM peptides in CKD-MBD predisposes the patients to VC because of the reduced inhibition of VC pathways). Potent inhibitors of ASARM-peptide bioactivity are provided that can block the function of ASARM.

Abnormalities in serum phosphorus, calcium, vitamin D3, FGF23 and PTH occur in CKD-MBD. The CKD-MBD systemic abnormalities accompany parathyroid gland hyperplasia, renal osteodystrophy (ROD) and mineral vascular or soft tissue calcification. Approximately 10% of the world population is affected by CKD-MBD with ROD and cardiovascular calcification contributing to high morbidity and mortality rates. Of major concern is the recent discovery of an association between CKD-MBD and cerebrovascular disease. Vascular calcification, renal osteodystrophy and hyperphosphatemia occurs progressively in CKD-MBD and are chiefly responsible for the mortality and morbidity. The clinical management of these changes are key in preventing vascular calcification (VC), ROD and related debilitating clinical sequelae, such as hypertension and left ventricular hypertrophy. Importantly, medial calcification of the aortae and calcification of the heart valves are directly associated with mortality and severe morbidity. Infusion of ASARM peptide in 56NEPHREX rats reduces serum phosphate levels, corrects renal osteodystrophy, suppresses brain, renal and cardiovascular calcification and prevents calciphylaxis like lesions in CKD-MBD and NSF.

In some embodiments, the ASARM is administered to bind with crystals or minerals in the subject. The ASARM can bind with nanocrystals of minerals to inhibit accumulation of crystals in a tissue, such as the kidney. The inhibition of crystallization inhibits loss of nephrons in the kidney. Therefore, renal function in the kidney can be retained or improved from the ASARM.

In some embodiments, a method of treating a kidney disorder can include: providing an ASARM peptide; and administering a therapeutically effective amount of the ASARM peptide to a subject to treat the kidney disorder. In some aspects, the method is for treating CKD-MBD by administering a therapeutically effective amount of the ASARM peptide to a subject to treat the CKD-MBD. In some aspects, the method is for treating calciphylaxis by administering a therapeutically effective amount of the ASARM peptide to a subject to treat the calciphylaxis. In some aspects, the method is for treating or preventing NSF by administering a therapeutically effective amount of the ASARM peptide to a subject to treat the NSF. In some aspects, the methods are performed by administering a therapeutically effective amount of the ASARM peptide to a subject to treat the condition by treating or inhibiting abnormalities in calcium, phosphorus, parathyroid hormone (PTH), vitamin D metabolism, bone and vascular calcification as well as atherosclerotic plaque deposition. In some aspects, the method includes administering a therapeutically effective amount of the ASARM peptide to a subject to treat a condition of ulcerative skin lesions. In some aspects, the method includes administering a therapeutically effective amount of the ASARM peptide to a subject to treat the fibrosing ulcerative indurations and red bumps. In some aspects, diabetic kidney diseases are treated and/or inhibited.

In some embodiments, a method of inhibiting phosphate update from a gut of a subject is provided. Such a method can include administering a therapeutically effective amount of the ASARM peptide to a subject to inhibit phosphate update from the gut of the subject. That is, the ASARM prevents phosphate from entering the blood. This can be used to treat hyperphosphatemia, such as by reversing hyperphosphatemia in the subject. The phosphate uptake can also be inhibited from the tubules of the kidney into the tissue thereof. As such, the ASARM can inhibit phosphate from going into or accumulating in the kidney tissue. This can inhibit hyperphosphatemia. It may also increase excretion of phosphates in urine.

In some embodiments, a method of treatment can include administering a therapeutically effective amount of the ASARM peptide to a subject to reduce soft tissue calcification in the subject. The soft tissue can be vascular, renal, liver, brain, heart, lung, skin, or other soft tissue. The reduction can be by reducing an amount or rate of calcification, as well as inhibiting calcification from occurring. The ASARM can provide a function that reduces formation of mineral deposits, such as calcium. As such, plaques of mineral deposition can be inhibited. This can be used to be renal-protective in the treatments and prophylaxes described herein.

In some aspects, the ASARM can bind with the minerals, such as calcium or hydroxyapatite, or other to inhibit crystal formation, which can inhibit nanocrystals. The ASARM can bind with the nanocrystals to prevent deposition or further crystallization. This can inhibit formation of crystal deposits or other mineralization of the tissues as described herein. The binding to crystals stops the crystals from growing or otherwise increasing in size, which can inhibit injury to the nephrons. The binding of ASARM to crystals can inhibit the deposition of the crystals in the kidney, and also inhibit the transition of vascular smooth muscle cells to osteoprogenitor cells. The inhibition of the transition can be by inhibiting microcrystal formation, which inhibits the transition.

In some embodiments, a method of correcting osteodystrophy in a subject is provided. Such as method can include administering a therapeutically effective amount of the ASARM peptide to a subject to correct osteodystrophy in the subject. The osteodystrophy may be renal osteodystrophy that is inhibited. The treatment may include administering the ASARM peptide to balance calcium, PTH, and activated vitamin D The treatment can inhibit bone turnover and changing bone volume. Also, the treatment can be a prophylaxis to inhibit osteodystrophy from developing or progressing. In some aspects, the administration for treatment of osteodystrophy is subdermal, where the deposition occurs under the skin. Intravenous administration, such as bolus or infusion, can also be used, which can be for any administration for any treatment.

In some embodiments, a method of preventing or inhibiting onset of calciphylaxis in a subject is provided. The method can include administering a therapeutically effective amount of the ASARM peptide to a subject without calciphylaxis to prevent or inhibit onset of calciphylaxis in the subject, wherein the ASARM peptide is administered as a prophylactic. As such, the subject may have an indication of being predisposed or susceptible to calciphylaxis, and thereby the ASARM is provided as a prophylactic. For example, the subject may have an indication of kidney disease or tissue calcification but without lesions, and the ASARM can be administered to inhibit onset of the lesions, and thereby inhibit onset of calciphylaxis.

In some embodiments, the ASARM is administered in any manner to obtain the therapeutic result. In some aspects, the ASARM peptide is administered orally, by injection (e.g., subcutaneous, intravenous, intraperitoneal, etc.), transdermally, or the like. For example, the method can include infusion of the ASARM peptide into the patient. In some aspects, the infusion is continuous infusion or intermittent infusion over a time period. Each rat (weight 250 g) received 3 mg of peptide over 28 days at an infusion rate of 4.46 ug/hour). This works out as 18.6 μg of ASARM-peptide kg⁻¹·hr⁻¹ (8.44 nmol kg⁻¹·hr⁻¹ or 5.1×10¹⁵ molecules kg⁻¹ hr⁻¹) for 28 days. In some aspects, the infusion is at an infusion rate of about 8.4 nmols kg⁻¹·hr⁻¹, or about 0.5 nmols kg⁻¹·hr⁻¹ to 50 nmols kg⁻¹·hr⁻¹, or about 1 nmols kg⁻¹·hr⁻¹ to about 25 nmols kg⁻¹·hr⁻¹, or about 5 nmols kg⁻¹·hr⁻to about 10 nmols kg⁻¹·hr⁻¹. In some aspects, the infusion is at an infusion rate of about 5.1×10¹⁵ molecules kg⁻¹·hr⁻¹, or from 0.5 to about 50×10¹⁵ molecules kg⁻¹·hr⁻¹, or about 1 to about 25×10¹⁵ molecules kg⁻¹·hr⁻¹, or about 5 to about 10×10¹⁵ molecules kg⁻¹·hr⁻¹.

In some embodiments, a method is provided for preventing GBCA (Gadolinium Binding Contrast Agent) induced calcification or other mineralization with the ASARM peptide. As such, the ASARM can be administered to a subject that has already been administered GBCA. After the GBCA is administered, the ASARM can be used as a prophylactic or treatment to inhibit mineral deposition or other GBCA induced mineralization of a tissue of the subject.

In some embodiments, a method of preventing or inhibiting onset of tissue calcification, such as brain calcification, is provided. The tissue can be any soft tissue described herein, where the brain is used as an example. The method can include administering a therapeutically effective amount of the ASARM peptide to a subject without brain calcification to prevent or inhibit onset of brain calcification in the subject, wherein the ASARM peptide is administered as a prophylactic. The CT imaging of the brain shows ASARM inhibited brain calcification or mineral deposition. For example, the older a person gets, the more likely mineral deposition, such as in the brain, will occur. Now, the ASARM peptide can inhibit such mineralization of tissues, such as the brain. The ASARM can inhibit mineralization and thereby can be protective as a prophylactic to mineralization. The inhibition of mineralization of the brain can inhibit cognitive decline from aging, and also be used to improve cognition. In some aspects, the method of treating or reducing brain calcification in a subject can include administering a therapeutically effective amount of the ASARM peptide to a subject treat or reduce brain calcification in the subject, wherein the ASARM peptide is administered as a prophylactic.

In some embodiments, the subject is human and has a diagnosis of a mineralization deposition disorder and/or a kidney disorder. In some aspects, the subject has CKD-MBD. In some aspects, the subject has a renal disease. In some aspects, the subject undergoes kidney dialysis as part of a therapeutic regimen. In some aspects, the subject is older than 45 years old. In some aspects, the subject is older than 55 years old. In some aspects, the subject is older than 65 years old. In some aspects, the subject is older than 75 years old.

### EXAMPLES

Synthetic ASARM peptide was administered to rats that had undergone subtotal 5/6th nephrectomy (56NEPHREX, 56NEPHX), which is a rodent model of CKD-MBD. All rats were fed a high phosphate diet (2% Pi) to worsen mineral metabolism defects. The effects of ASARM infusion in rats exposed to gadolinium contrast agents (Omniscan™) was also studied. Changes in serum potassium, phosphate, blood urea nitrogen (BUN), creatinine, PTH, FGF23 and calcium excretion were assessed in response to 28 days of ASARM peptide infusion. Also, changes in bone quality, soft tissue calcification and expression of gut Npt2b (Slc34a2) were studied following ASARM peptide treatment.

The 56NEPHREX rats treated with ASARM peptide showed significant improvements in reduced hyperphosphatemia and BUN compared to vehicle controls. Also, ASARM infused 56NEPHREX rats displayed reduced renal, bone, brain and cardiovascular calcification. Notably, ASARM-peptide infusion prevented the genesis of sub-dermal medial blood vessel calcification and calciphylaxis like lesions in 56NEPHREX rats compared to 56NEPHREX rats infused with vehicle. Also, massive sub-dermal calcifications and ulcerative NSF-like skin lesions occurring in 56NEPHREX rats exposed to the gadolinium contrast agent Omniscan™ were prevented by ASARM peptide infusion. Thus, ASARM peptide infusion corrects hyperphosphatemia and improves bone, renal, vascular and skin mineralization abnormalities in 56NEPHREX rats. These findings confirm the utility of ASARM peptide treatment in patients with CKD-MBD, calciphylaxis, and NSF as well as reduce VC.

Experimental Protocol

To show ASARM can treat kidney disease, a Wister rat subtotal 5/6th nephrectomy experimental CKD-MBD model (NEPHREX) was used and sham operated rats (SHAM) were used as controls. Male rats (16 week, 250 gm) were fed a high phosphate diet to worsen mineral metabolism defects (2% P, 2000 IU Vit D and 0.8% Ca; Teklad #170496; Envigo envigo.com/teklad). Protocol and rat numbers for osmotic pump (Alzet) infusion of ASARM-peptide, and intravenous injection are determined. Pump infusion with ASARM peptide or vehicle was carried out for 28 days and rats were maintained on a high phosphate diet. In the timeline of experiment, 3× intravenous injections of Omniscan™ (gadolinium contrast agent—gadodiamide, Novaplus®) were administered via jugular vascular catheter on days 12, 13 and 14 on two additional groups of rats that were treated with vehicle or ASARM peptide.

Infusion of ASARM:

Ten days after 5/6 nephrectomy or sham operations, rats were subjected to the following protocols. There were 6 SHAM operated rates with no ASARM, but with vehicle and high phosphate diet. There were 10 CKD-MBD model rates administered ASARM without a vehicle, but with high phosphate diet. There were 12 CKD-MBD model rates without ASARM, but with vehicle and high phosphate diet. The biological samples of blood and urine were taken from the rats at 5 days post-surgery, blood was taken at 12 days. At day 12, the infusion of ASARM began, with blood and urine being collected at day 24, blood being taken at day 35, and then blood, urine, and necropsy at day 47.

Continuous infusion (28 days) of ASARM peptide and vehicle was undertaken using subcutaneous transplantation of alzet osmotic-pumps (Durect Corporation, Cupertino Calif.). Pump models 2ML4 (28 day model; infusion rate 2.5 μL/hr) were used. The pumps were implanted in rats anesthetized with isoflurane. An ASARM peptide pump infusion rate of 8.4 nmols/kg/hr or 5.1×10¹⁵ molecules/kg/hr) was used for 28 days. This flow rate is based on prior results and consistent with known circulating levels of ASARM-peptides in HYP and wild type rodents. Animals were housed individually, and food intake monitored to assess whether treatments affect appetite. The diet (TEKLAD #170496) was high in non-phytates inorganic phosphate (Pi). Specifically, the high phosphate diet (HPO₄) consist of 2% Pi and 2000 U of vitamin D (Teklad, 170496). Note 3× iv injections of Omniscan™ (gadolinium contrast agent—gadodiamide, Novaplus®) were administered via jugular vascular catheter (JVC) on days 12, 13 and 14 on two groups of rats that were treated with vehicle or ASARM peptide. JVC's were surgically implanted and allowed direct iv injections of Omniscan™ at 6 mmol/kg/injection.

Serum and Urine Analysis:

Serum Pi, calcium, fractional excretion of phosphate, FGF23, KLOTHO, PTH, 1,25 Vit-D3, osteopontin, immunohistology, MEPE and analyses were carried out as described previously [Zelenchuk, L V, et al., Age dependent regulation of bone-mass and renal function by the MEPE ASARM-motif. Bone, 79: 131-142, 2015; Zelenchuk, L, et al., SPR4-peptide Alters Bone Metabolism of Normal and HYP Mice. Bone, 72: 23-33, 2015; Zelenchuk, L V, et al., PHEX Mimetic (SPR4-Peptide) Corrects and Improves HYP and Wild Type Mice Energy-Metabolism. PLoS One, 9: e97326, 2014; David, V, et al., ASARM peptides: PHEX-dependent & independent regulation of serum phosphate. Am J Physiol Renal Physiol, 300: F783-791, 2011; David, V, et al, Matrix extracellular phosphoglycoprotein (MEPE) is a new bone renal hormone and vascularization modulator. Endocrinology, 150: 4012-4023, 2009; Martin, A, et al, Degradation of MEPE, DMP1, and release of SIBLING ASARM-peptides (minhibins): ASARM-peptide(s) are directly responsible for defective mineralization in HYP. Endocrinology, 149: 1757-1772, 2008; Rowe, P S, et al, Correction of the mineralization defect in hyp mice treated with protease inhibitors CA074 and pepstatin. Bone, 39: 773-786, 2006; Rowe, P S N, et al, Surface Plasmon Resonance (SPR) confirms MEPE binds to PHEX via the MEPE-ASARM-motif: A model for impaired mineralization in X-linked rickets (HYP). Bone, 36: 33-46, 2005; Rowe, P S, et al., Do ASARM peptides play a role in Nephrogenic Systemic Fibrosis? Am J Physiol Renal Physiol, 309: F764-769, 2015.] ASARM peptides in sera, urine and culture media were measured using our extensively published and developed Enzyme Linked Immunosorbent Assays (ELISA's) [id; Bresler, D, et al., Serum MEPE-ASARM-peptides are elevated in X-linked rickets (HYP): implications for phosphaturia and rickets. J Endocrinol, 183: R1-9, 2004; Yuan, B, et al, Aberrant Phex function in osteoblasts and osteocytes alone underlies murine X-linked hypophosphatemia. J Clin Invest, 118: 722-734, 2008].

The ASARM motif is highly conserved and occurs in a class of proteins that all map to a clustered region on chromosome 5. These proteins are called (SIBLINGs) “small integrin-binding ligand, N-linked glycoproteins.” The SIBLINGs include MEPE, DMP1, DSPP, Osteopontin, Bone Sialo protein and Statherin. Although not reported it is possible the related “SIBLING” ASARM peptides (released from ASARM motifs) have similar positive effects (e.g., studies used the MEPE ASARM motif). As such, the ASARM can be substituted with any SIBLING protein.

Micro-Computed Tomography (μCT)

Femurs, kidneys, heart, ascending aortae and skin were removed at necropsy screened using a μCT40 Scanco system as described previously [id]. For vasculature analyses, rats were perfused with MicroFil™, brains and tissues were then scan by μCT40 imaging system. Rat brain anatomic structure and mineral composition were defined using Omnipaque™ iodine contrast treatment and μCT scanning

Results

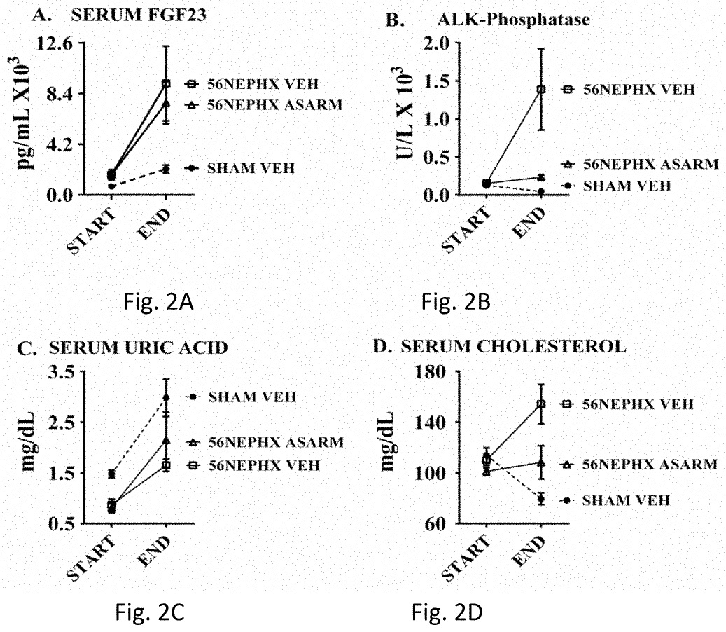

It was found that ASARM prevents hyperphosphatemia and corrects renal function. Abnormally high serum phosphate levels occurred with 56NEPHREX rats at the start of the study and prior to vehicle or ASARM peptide infusion. The hyperphosphatemia increased dramatically with the vehicle treated 56NEPHREX rats as the experiment progressed over 28 days of infusions. In contrast, increased hyperphosphatemia was prevented completely in 56NEPHREX (referenced as abbreviation 56NEPHX in figures) rats that received ASARM peptide (FIGS. 1A-1L). The corrected hyperphosphatemia occurred even though serum FGF23 levels remained high with the ASARM treated 56NEPHREX rats (FIG. 2A-2D). Similar positive effects occurred with serum creatinine, BUN, potassium, calcium, bicarbonate and protein (FIGS. 1A, 1B, 1C, 1D, 1E, 1G, and 1I). A striking improvement in serum alkaline phosphatase also occurred with ASARM treated 56NEPHREX rats compared to vehicle treated controls (FIG. 2B). Because of the high phosphate diet (2% Pi; HPO₄), high serum PTH levels also occurred with SHAM and 56NEPHREX rats treated with vehicle or ASARM peptide (FIG. 1J). Although hypocalcemia was significantly improved with the 56NEPHREX ASARM treated rats compared to vehicle, both 56NEPHREX groups presented with significant hypocalcemia compared to the SHAM rats. Increased serum uric acid levels occurred for all groups after 28 days with no significant differences (FIG. 2C). Serum cholesterol levels increased strikingly with SHAM rats and a marked hypocholesterolemia occurred with the 56NEPHREX vehicle treated rats over 28 days. In contrast, the ASARM treated 56NEPHREX rats serum cholesterol remained unchanged over the 28 days (FIG. 2D).

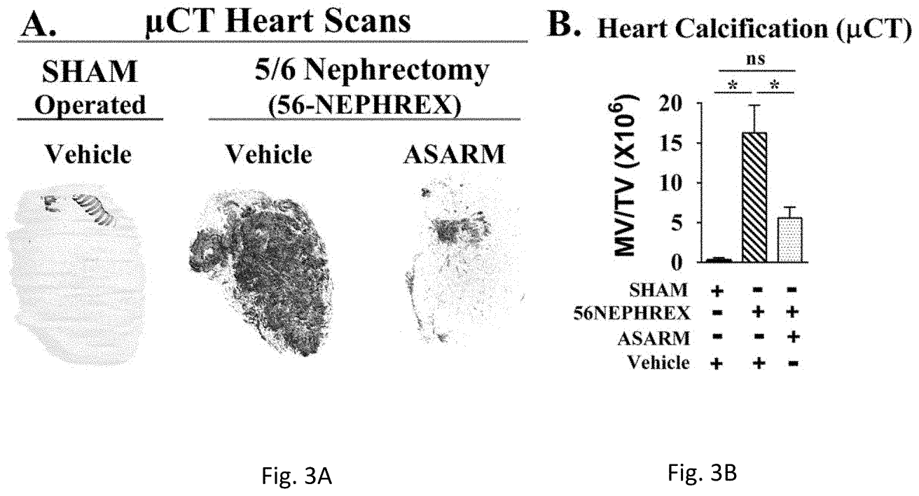

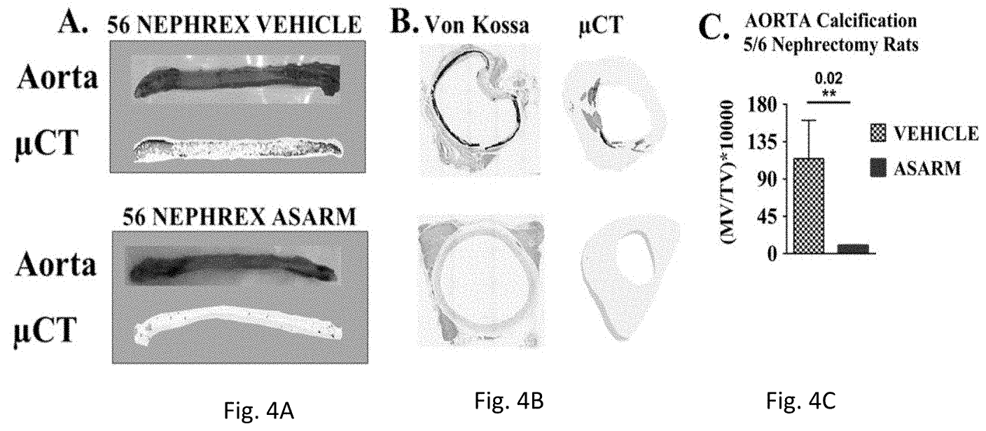

It was found that the ASARM peptide prevents calcification of the heart and medial calcification of the aorta. Measurements of calcification of the hearts and aortae using μCT and Von Kossa staining of histology sections revealed major improvements with ASARM. The HPO₄ diet caused severe cardiac calcification with 56NEPHREX rats infused with vehicle (FIGS. 3A and 3B). With SHAM rats, no cardiac calcification was observed. Low level tracheal calcification occurred with all three groups. A significant and dramatic reduction in cardiac calcification occurred with 56NEPHREX rats infused with ASARM peptide when compared to vehicle treated 56NEPHREX rats (FIGS. 3A-3B). Severe medial calcification of the aorta also occurred with 56NEPHREX rats infused with vehicle. With SHAM rats, no calcification of the aorta occurred (FIGS. 4A-4C). A significant and dramatic reduction in medial calcification of the aortae occurred with the 56NEPHREX rats infused with ASARM peptide (FIGS. 4A-4C). The dramatically suppressed medal calcification of the aortae in ASARM treated 56NEPHREX rats is consistent with the observed suppression of cardiac calcification (FIGS. 3A-3B and 4A-4C).

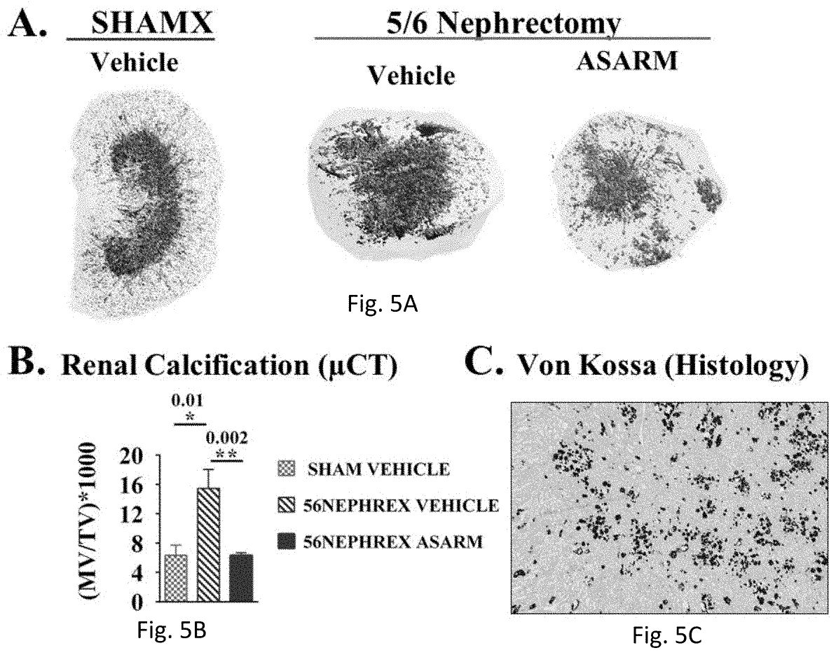

It was found that ASARM peptide suppresses renal calcification in CKD-MBD rats. Kidneys removed at necropsy after 28 days were scanned using μCT and Von Kossa staining of histological sections. Significant calcification of the kidneys occurred in SHAM and both 56NEPHREX groups fed the HIPO₄ diet (FIGS. 5A-5C). Consistent with the improved renal function observed in 56NEPHREX rats treated with ASARM peptide a significant reduction in renal calcification occurred (FIGS. 5A-5C). Given ASARM peptide corrected the hyperphosphatemia the decrease in renal calcification is a significant finding and is consistent with the observed improved renal function (FIGS. 1A, 1C, 1D and FIGS. 5A-5C).

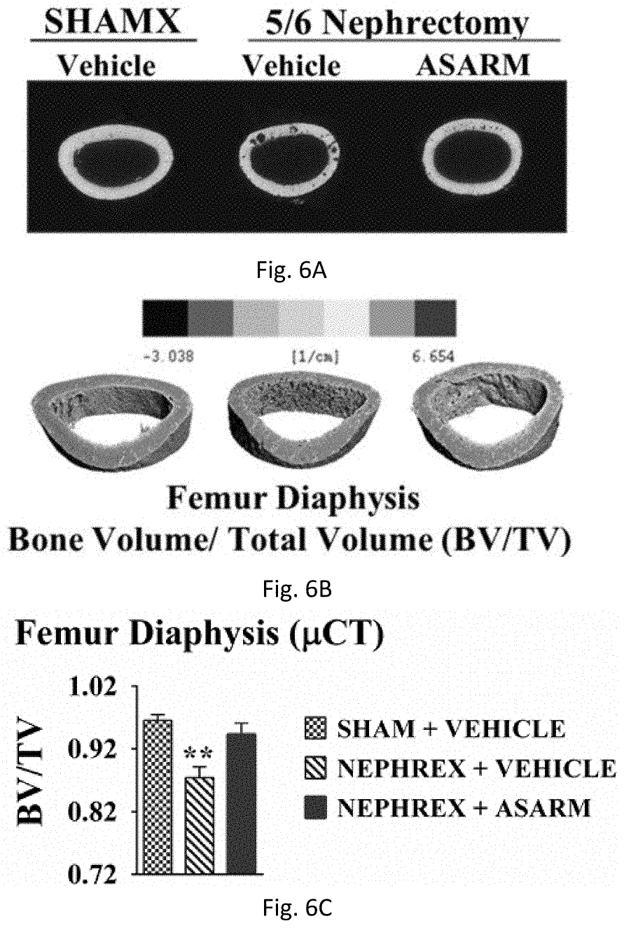

It was found that ASARM peptide corrects bone abnormalities in CKD-MBD rats. The 56NEPHREX rats that were fed a high phosphate diet (HPO₄) developed significant bone mineralization defects with reduced bone volume/total volume ratios (BV/TV; FIGS. 6A-6C). Also, consistent with progressive renal failure and CKD MBD sequelae a significantly increased serum alkaline phosphatase occurs with 56NEPHREX vehicle treated rats (FIG. 2B). In contrast, significant corrections in bone quality and serum alkaline phosphatase levels occurred with 56NEPHREX rats infused with ASARM peptide (FIG. 2B and FIGS. 6A-6C). In combination the data shows ASARM peptides correct the systemic, mineral cardiovascular and bone abnormalities in a rat model of CKD-MBD. These three pathologies represent the defects occurring in chronic disease-mineral bone disease.

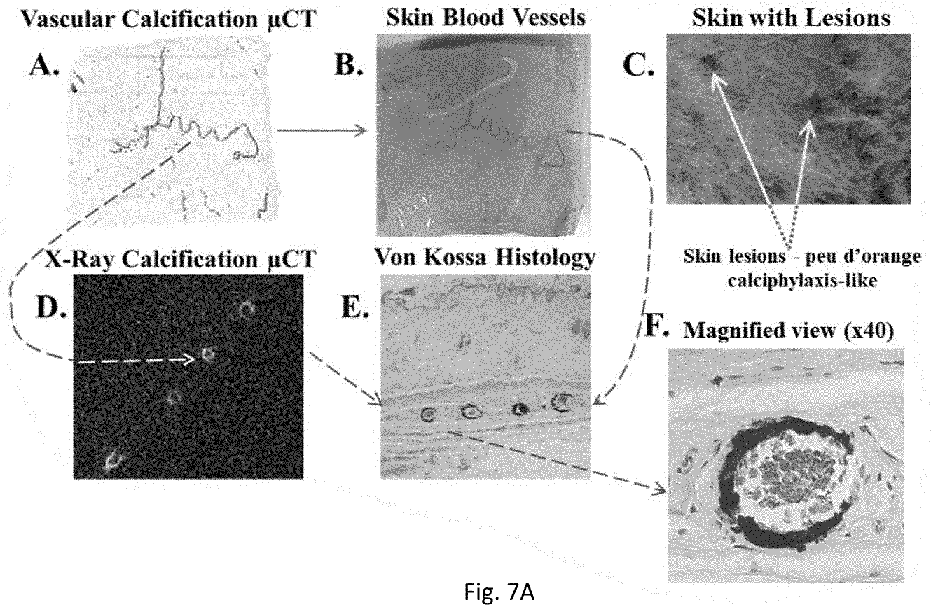

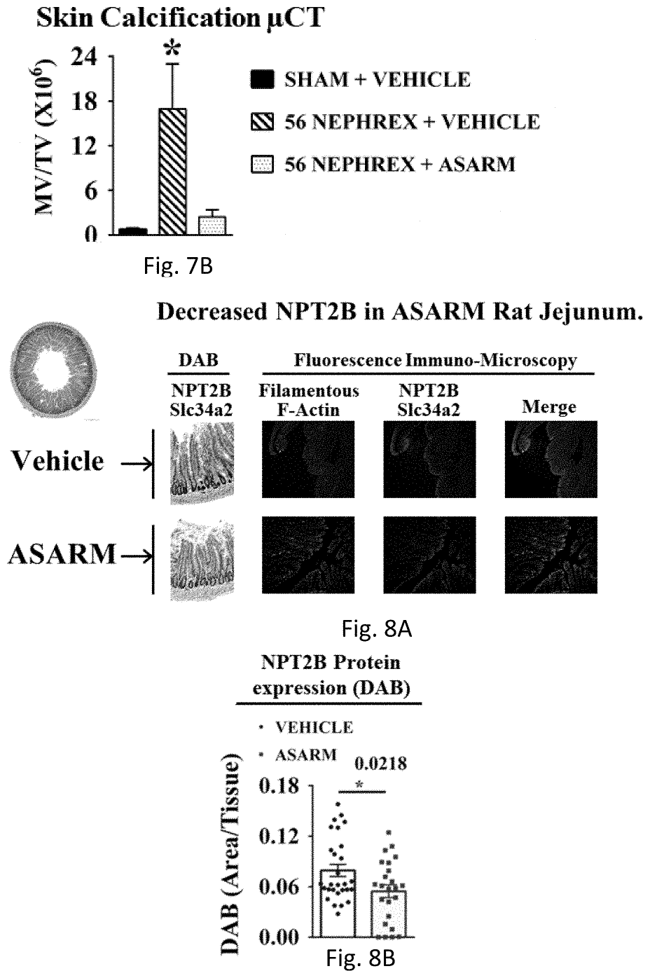

It was found that ASARM peptide suppresses calciphylaxis-like lesions in CKD-MBD rats. The HIPO₄ diet used in this study (2% PO4; non phytates) induced prominent skin abnormalities that appeared as “peau d'orange”, pre-ulcerative lesions in vehicle treated 56NEPHREX rats (FIGS. 7A-7B). The lesions were characteristic of skin calciphylaxis and occurred on the flank, abdominal and rear area (FIG. 7A, Panel C). Some rats developed lesions on top of the head or scalp. Strikingly, no lesions were detected with SHAM rats treated with vehicle or 56NEPHREX rats treated with ASARM peptide. Analysis of 1 cm² sections of skin sections by μCT and histology showed heavy medial mineralization of skin blood vessels (FIGS. 7A-7B).

It was found that ASARM peptide suppresses rat jejunum NPT2B phosphate transporter expression. Progressive systemic and hormonal changes in CKD-MBD should result in a reduced fractional intestinal phosphorus absorption that compensates for impaired renal phosphorus excretion. However, CKD animal models show a failure of the intestines to compensate for hyperphosphatemia. Indeed, intestinal phosphate uptake is not statistically different between CKD rats and controls with no change in NPT2B mRNA expression [Vorland, C J, et al., Disease Progression Does Not Decrease Intestinal Phosphorus Absorption in a Rat Model of Chronic Kidney Disease-Mineral Bone Disorder. J Bone Miner Res, 2019; Marks, J, et al., Intestinal phosphate absorption in a model of chronic renal failure. Kidney Int, 72: 166-173, 2007]. A marked correction in CKD associated hyperphosphatemia occurs in “adenine-induced” uremic Npt2b-knockout mice compared with uremic wild-type control mice, which showed an improved survival of the uremic Npt2b knockout mice compared to uremic wild-type mice [Schiavi, S C, et al., Npt2b deletion attenuates hyperphosphatemia associated with CKD. J Am Soc Nephrol, 23: 1691-1700, 2012.]. The studies suggest synthetic ASARM peptide is a useful treatment for the intestinal maladaptation by inhibiting intestinal phosphate uptake and vascular calcification (FIG. 8 in view of FIGS. 3A-3B, 4A-4C, and 7A-7B). It has been shown that administration of MEPE protein or ASARM peptide inhibits renal and intestinal phosphate. ASARM inhibition of phosphate-uptake occurs in rats and mice in vitro, ex vivo and in vivo [David, V, et al., ASARM peptides: PHEX-dependent & independent regulation of serum phosphate. Am J Physiol Renal Physiol, 300: F783-791, 2011; Martin, A, et al., Degradation of MEPE, DMP1, and release of SIBLING ASARM-peptides (minhibins): ASARM-peptide(s) are directly responsible for defective mineralization in HYP. Endocrinology, 149: 1757-1772, 2008; Rowe, P S, et al., MEPE has the properties of an osteoblastic phosphatonin and minhibin. Bone, 34: 303-319, 2004; Shirley, D G, et al., Direct micropuncture evidence that matrix extracellular phosphoglycoprotein inhibits proximal tubular phosphate reabsorption. Nephrol Dial Transplant, 25: 3191-3195, 2010; Marks, J, et al., Matrix extracellular phosphoglycoprotein inhibits phosphate transport. J Am Soc Nephrol, 19: 2313-2320, 2008; Dobbie, H, Unwin, R J, Faria, N J, Shirley, D G: Matrix extracellular phosphoglycoprotein causes phosphaturia in rats by inhibiting tubular phosphate reabsorption. Nephrol Dial Transplant, 23: 730-733, 2008; Marks, J, et al., Matrix Extracellular Phosphoglycoprotein (MEPE) acutely inhibits renal and intestinal phosphate transport absorption (Abstract). Journal of the American Society for Nephrology, 18: SU-PO766, 2007]. Now, for the first time ASARM-peptide (2.2 kDa) specifically inhibits “intestinal phosphate” uptake (FIGS. 8A-8B). Thus our research has discovered the defined protein sequence responsible for the inhibition of intestinal phosphate uptake (AS ARM peptide).

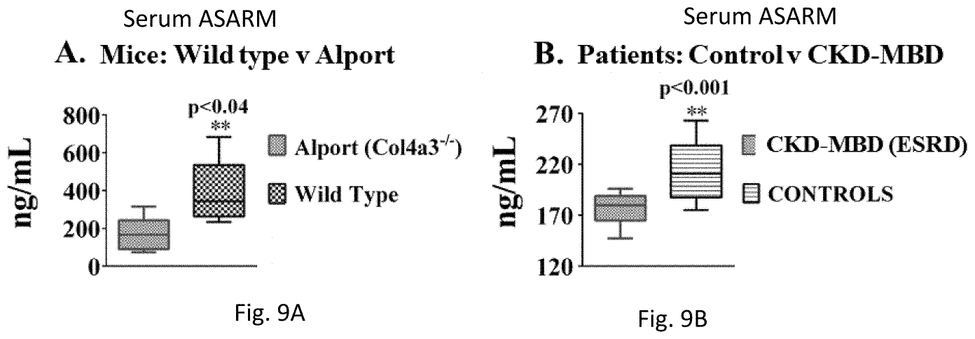

In has been found that that Alport mice and end stage renal disease patients have decreased serum ASARM peptide. ASARM peptides derived from osteocyte expressed extracellular matrix SIBLING proteins (MEPE and DMP1) play a key role in mineralization and FGF23 expression. In CKD-MBD down regulation of DMP1 and MEPE occurs and correlates with FGF23. Notably, transgenic DMP1 mice crossed with Alport mice (Col4a3^{-/-}/trgDMP1) show corrected CKD-MBD abnormalities [Dussold, C, et al., DMP1 prevents osteocyte alterations, FGF23 elevation and left ventricular hypertrophy in mice with chronic kidney disease. Bone Res, 7: 12, 2019]. Using Enzyme Linked Immunosorbent Assay (ELISA) we measured circulating ASARM peptides in end stage renal disease patients (increased FGF23) and Alport mice (an established murine CKD-MBD model). In both cases a significant decrease in ASARM peptides occurred compared to healthy, gender and age matched controls (FIGS. 9A-9B).

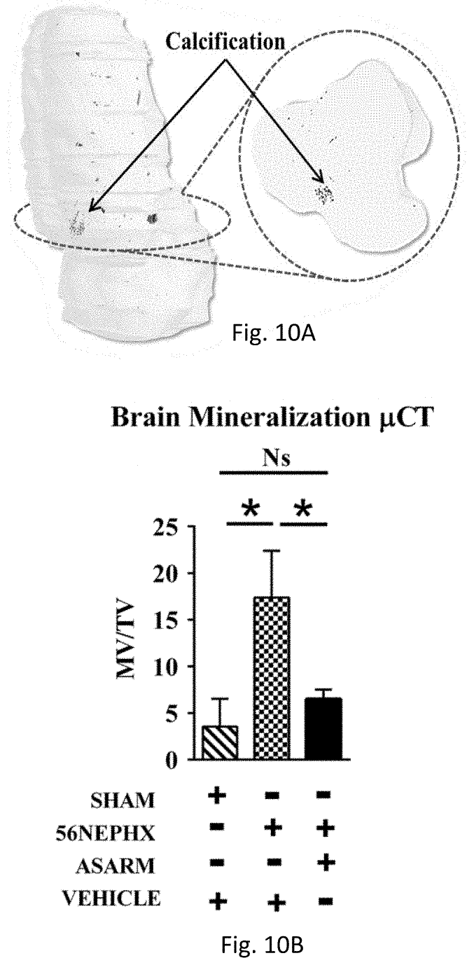

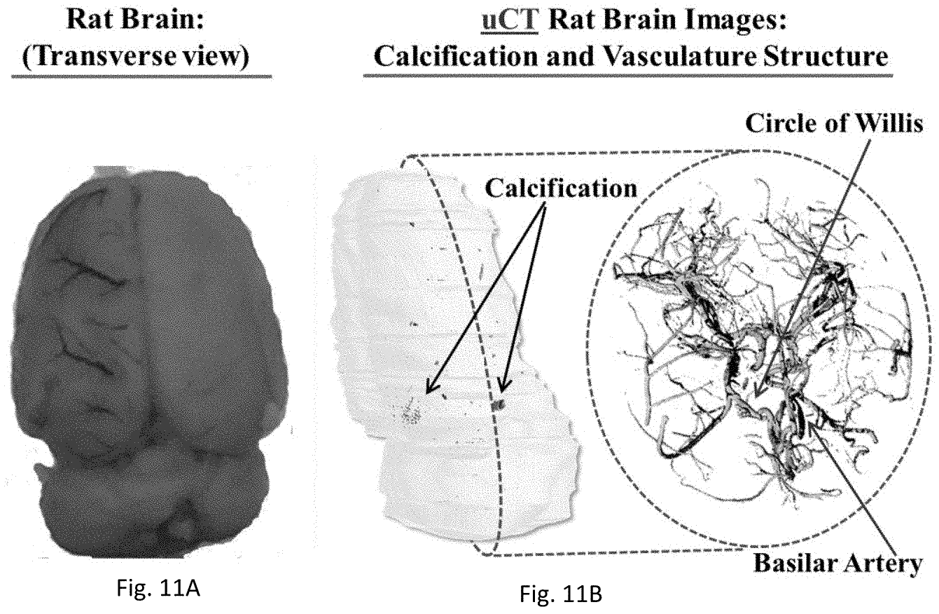

It has been found that brain calcification and abnormal vasculature (e.g., VC) in CKD-MBD rats is corrected with ASARM peptide. Accordingly, ASARM infusion prevents mineral deposition in CKD-MBD rat brains. Vehicle treated 56-NEPHREX rats developed brain calcifications (FIGS. 10A-10B and 11A-11B). Several calcified regions were identified using Allen's Brain Atlas these included the globus pallidus, dentate nucleus, pons, putamen, superior colliculus, primary motor area, angular insular area, entorhinal area, vermal regions and frontal lobes. Using an iodine contrast agent (Omnipaque™) and μCT 3D high resolution (6 μM) imaging regions of calcification were mapped to specific brain structures. Sham operated and ASARM treated NEPHREX rats showed major and significant reductions of brain mineral deposits (FIGS. 10A-10B and 11A-11B).

The rats had abnormal brain vasculature in the rat CKD-MBD model. Anatomic vasculature structure was visualized in 3D using μCT scans of MicroFil perfused rats (FIGS. 11A-11B). The number of blood vessels, diameter, connectivity, anisotropy and structural modeling can be determined using this technique. The studies show 56NEPHREX rats treated with ASARM peptide have improved vasculature compared to 56NEPHREX rats treated with vehicle. Vascular changes will impact brain anatomic structures and ASARM peptide treatment will likely improve cognitive outcomes in CKD-MBD patients.

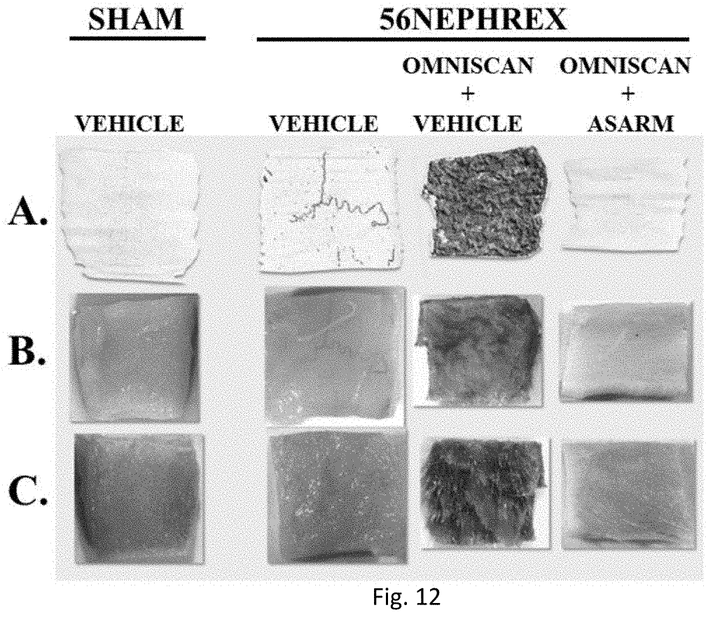

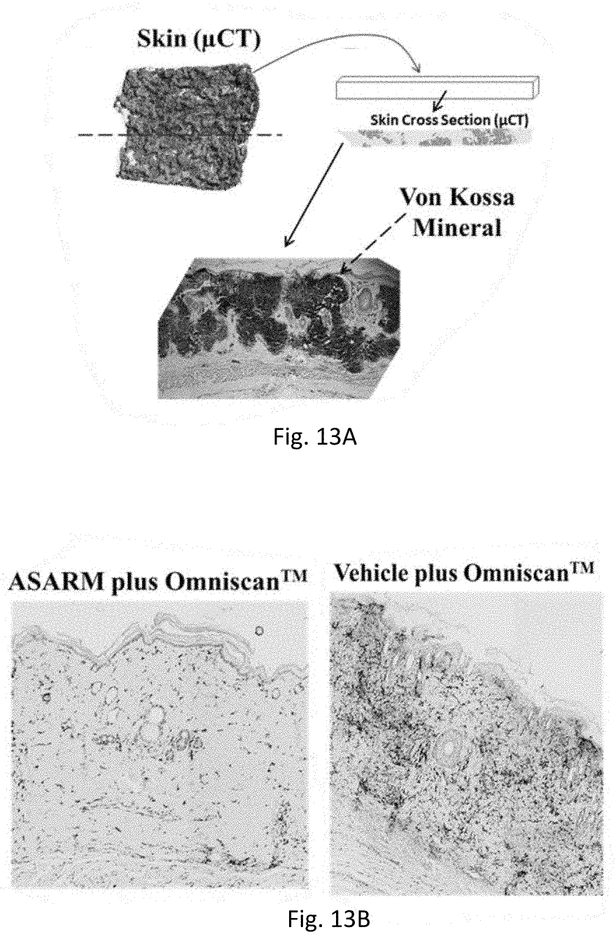

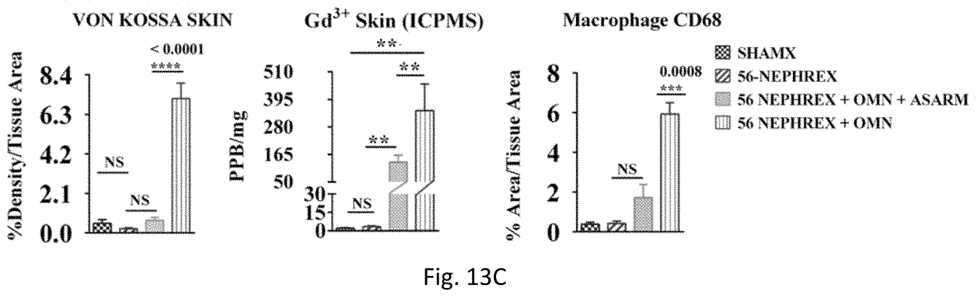

It was found that ASARM infusion prevents NSF in 56NEPHREX rats exposed to gadolinium contrast agent. In separate experiments, the data showed ASARM peptide infusion is strikingly effective at reversing NSF sequelae in 56NPHREX rats exposed to gadolinium contrast agent, Omniscan™. Male rats (age 6 weeks and weight 250 gm) were fed a high phosphate diet (HPO₄) to worsen mineral metabolism defects as described for the preceding experiments (2% P, non-phytates, Teklad.170496). ASARM peptide was infused into 56NEPHREX rats for 28 days using subcutaneous alzet osmotic pump implants. ASARM peptide was infused at a rate of 8.4 nmols/kg/hr or 5.1×10¹⁵ molecules/kg/hr, also as described for preceding experiments. For controls, sham operated rats (SHAM) and 56NPHEX rats were infused with vehicle (N=6). There were severe NSF-like skin lesions in 56NPHEX rats fed a high phosphate diet that were injected via implanted jugular vascular catheters (JVC) with Omniscan™ (data not shown). Specifically, three Omniscan™ injections were given consecutively on days 22, 33 and 24 “post-surgery” at 6 mmol/kg/injection. As described herein, the high phosphate diet (HIPO₄) used in this study (2% PO₄; non phytates) induced prominent skin abnormalities that appeared as peau d'orange, pre-ulcerative lesions in vehicle treated 56NEPHREX rats. The calciphylaxis lesions were strikingly worse in the 56NEPHREX rats treated with Omniscan™. Massive and extended calcification lesions occurred on the flank, abdominal and rear area. Some rats developed lesions on top of the head or scalp and the μCT scans appeared as “crocodilian-like” sheets of mineralized sub dermal deposits (FIG. 12). Strikingly, no lesions were detected with SHAM rats treated with vehicle and more significantly 56NEPHREX rats treated with ASARM peptide (FIG. 12). Analysis of 1 cm² sections of skin sections by μCT and histology showed heavy medial mineralization of skin blood vessels (FIGS. 12 and 13A-13C). Also, immunohistochemistry revealed extensive increases in inflammatory markers and macrophage recruitment (FIGS. 13A-13C).

Treatment and Compositions

Acidic, serine- and aspartic acid-rich MEPE associated (ASARM) [Rowe P S, de Zoysa P A, Dong R, Wang H R, White K E, Econs M J, Oudet C L. MEPE, a new gene expressed in bone marrow and tumors causing osteomalacia. Genomics. 2000; 67(1):54-68. Epub Aug. 17, 2000. doi: 10.1006/geno.2000.6235. PubMed PMID: 10945470] peptides occur naturally and originate from conserved AS ARM motifs located on a family of bone matrix proteins called small integrin-binding ligand, N-linked glycoproteins (SIBLINGs)

The peptide sequence of ASARM is: NH₂-RDDSSESSDSGS-(PO₃H₂)SS-(PO₃H₂)ES-(PO₃H₂)DGD-OH (SEQ ID NO: 1).

This ASARM peptide may be used as shown, or it may be linked on the C-terminal end and/or N-terminal end to other peptides or chemical moieties. Peptide purity was greater than 90% via HPLC, ion-exchange and also mass spectrometry.

In one embodiment, a pharmaceutical composition can include: a pharmaceutical carrier; and a polypeptide in the pharmaceutical carrier and having a sequence that has at least 75% complementarity to or at least 75% identical to ASARM. In one aspect, the polypeptide has at least 80% complementarity to or is at least 80% identical to ASARM. In one aspect, the polypeptide has at least 90% complementarity to or is at least 90% identical to ASARM. In one aspect, the polypeptide has at least 95% complementarity to or is at least 95% identical to ASARM. In one aspect, the polypeptide has at least 99% complementarity to or is at least 99% identical to ASARM. In one aspect, the polypeptide has 100% complementarity to or is 100% identical to ASARM.

In one embodiment, the polypeptide is included in a fusion polypeptide with second polypeptide. The second polypeptide can be any polypeptide, which can be on the N- or C-terminus. The polypeptide can provide beneficial properties, such as water solubility, receptor targeting, endosomal escape, or any other benefit. In one aspect, the endosomal disrupting polypeptide includes PC4 or derivative thereof.

The technology also relates to variant forms of these sequences and/or of these fragments. The expression “variant” indicates a polypeptide or a peptide that differs, for example, from the sequence of a reference peptide while keeping its essential properties. Generally, the differences are limited so that the sequences of the reference peptide and those of the variant are quite similar and, in some regions, identical.

Preferentially, the variant forms are those which vary from reference sequences by the substitution of chemically equivalent (or homologous) amino acids, that is, by the substitution of a residue with another possessing the same characteristics. Thus, classical substitutions take place between Ala, Val, Leu and Ile; between Ser and Thr; between the acid residues Asp and Gln; and between the basic residues Lys and Arg, or between the aromatic residues Phe and Tyr.

The expression “variant” indicates a polypeptide or a peptide that differs, for example, from the sequence of a reference peptide while keeping its essential properties. Generally, the differences are limited so that the sequences of the reference peptide and those of the variant are quite similar and, in some regions, identical. A variant peptide and a reference peptide may differ in their amino acid sequence by one or several substitutions, additions, or deletions in all the combinations.

In the technology, the term “amino acid” refers to any natural or unnatural organic acid having the formula (II): —NHR—CR—C(O)—O (II), where each —R is independently selected from a hydrogen or an alkyl group having between 1 and 12 carbon atoms. Preferentially, at least an —R group of each amino acid is a hydrogen. The term “alkyl” refers to a carbon chain that can be linear or branched, substituted (mono- or poly-) or not substituted; saturated, mono-saturated (a double or triple bond in the chain), or poly-unsaturated (two or several double bonds, two or several triple bonds, one or several double bonds, and one or several triple bonds in the chain).

Many biologically compatible forms of protection can be considered, such as acylation or acetylation of the amino terminal end, or amidation or esterification of the terminal carboxyl end. Such forms are well known by those skilled in the art. Thus, the technology relates to the use as previously defined and is characterized by the fact that the peptide either is or is not in a protected form. Preferably, the protection used is either acylation or acetylation of the amino terminal group, or esterification or amidation of the terminal carboxyl end, or both of them. The amino acid derivatives and the peptide derivatives also relate to amino acids and peptides bound together by a pseudo-peptide bond. By the term “pseudo-peptide bond,” we refer to all types of bonds likely to replace “classical” peptide bonds.

In the domain of amino acids, the geometry of the molecules is such that they can be theoretically presented as different optical isomers. There is indeed a molecular conformation of the amino acid (AA) such that it deviates to the right of the plane of polarization of the light (dextrorotatory conformation or D-aa), and a molecular conformation of the amino acid (aa) such that it deviates to the left of the plane of polarization of the light (levorotatory conformation or L-aa). Nature retained for the natural amino acids only levorotatory conformation. Consequently, a peptide of natural origin will be made up only of amino acids of type L-aa. However, chemical synthesis in a laboratory makes it possible to prepare amino acids having two possible conformations. From this basic material, it is thus possible to incorporate, during peptide synthesis, amino acids in the form of dextrorotatory or levorotatory optical isomers. Thus, the amino acids constituting the peptide according to the technology, can be under configuration L- and D-; in a preferential way, amino acids are in L configuration. The peptide according to the technology can be in L, D, or DL-configuration.

According to the technology, the peptides can be prepared using all appropriate methods. Thus, the peptides can be isolated peptides from peptides and proteins existing naturally, recombinant peptides, synthetic peptides, or peptides produced by a combination of these methods. Of course, the methods, in order to prepare the peptides according to the technology, are well known by one skilled in the art. Thus, the peptide according to the technology may be of natural or synthetic origin. Preferentially, according to the technology, the peptide is obtained by chemical synthesis.

The compounds described herein can be used in pharmaceutical compositions for providing the treatments described herein. The compounds can be formulated for administration by any suitable route as described herein to a subject having or suspected of having a disorder that can be treated with ASARM peptide.

In a related aspect, a pharmaceutical composition is provided, the pharmaceutical composition including an effective amount of ASARM peptide (or pharmaceutically acceptable salt thereof) for treating a condition.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment of a disease or disorder. As used herein, a “subject” or “patient” is a mammal, such as a cat, dog, rodent or primate. Typically the subject is a human, and, preferably, a human suffering from or suspected of suffering from an addiction. The term “subject” and “patient” can be used interchangeably.

Thus, the instant present technology provides pharmaceutical compositions and medicaments comprising ASARM peptide, or derivative thereof, prodrug thereof, and optionally a pharmaceutically acceptable carrier or one or more excipients or fillers. ASARM peptide can be used as scaffold for derivatizing and preparing derivatives. The substituents and chemical moieties on the core scaffold structure may be substituted with other substituents, such as those on the other scaffolds, or those described herein or otherwise generally known.

The compositions may be used in the methods and treatments described herein. Such compositions and medicaments include a therapeutically effective amount of any compound as described herein. The pharmaceutical composition may be packaged in unit dosage form. The unit dosage form is effective in treating a disease or disorder when administered to a subject in need thereof.

The pharmaceutical compositions and medicaments may be prepared by mixing ASARM peptide with pharmaceutically acceptable carriers, excipients, binders, diluents or the like to prevent and treat disease or disorder. The compounds and compositions described herein may be used to prepare formulations and medicaments that prevent or treat a variety of disorders. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount, such as by simply administering a compound of the present technology to a patient in increasing amounts until the progression of the condition/disease state is decreased or stopped. The compounds of the present technology can be administered to a patient at dosage levels in the range of about 0.1 to about 100 mg per day, but possibly up to 500 to 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art. For example, the rats (e.g., weight 250 g) received 107 ug/day or 428 ug/kg/day. An equivalent dose for a 70 kg patient would be 29971 ug, or 29 mg.

Various assays and model systems can be readily employed to determine the therapeutic effectiveness of the treatment according to the present technology.

The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective for the treatment of a disease or disorder.

In one aspect, an ASARM peptide of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising ASARM peptide of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

According to an advantageous mode of embodiment of the technology, the abovementioned peptides are solubilized beforehand in one or several cosmetically or acceptable solvents classically used by one skilled in the art, such as water, ethanol, propylene glycol, butylene glycol, dipropylene glycol, ethoxylated or propoxylated diglycols, cyclic polyols, vaseline, a vegetal oil, or any combinations of these solvents.

In one embodiment, the polypeptide is dissolved in the carrier. In one aspect, the carrier includes one or more of cetearyl alcohol, cetearyl glucoside, squalane, isopropyl palmate, octyldodecaonol, phenoxyethanol, methylparaben, etheylparaben, butylparaben, propylparaben, isobutylparaben, glycerin, butylene glycol, sodium acrylate, acryloyldimethyl taurate, isohexadecane, polysorbate, glyceryl stearate, dicaprylyl ether, alkyl benzoate, isononyl isononanoate, methylpropanediol, tetrasodium EDTA, iodoproynyl butylcarbamate, triethanolamine, ketoconazole, serenoa serrulata extract, emu oil, niacin vitamin B3, caffeine, pyridoxine, L-pathenol, linolenic acid, simmondsia chinesis seed oil, zinc oxide, lecithin, ZnCl₂, L-α-phosphatidylcholine, ethanol, PBS, phospholipids, fatty acids, tocopherol, and derivatives thereof and equivalents thereof. In one aspect, the polypeptide is contained in a liposome or microsphere or polymer particle or lipid emulsion or combination thereof, and such can be included in a carrier.

According to another advantageous mode of embodiment of the technology, the abovementioned peptides are solubilized beforehand in one vector such as liposomes, emulsions, solid lipid nanoparticles, micelles, micro- or nano-particles or adsorbed on powdery organic polymers, mineral supports like talcs and bentonites, and more generally solubilized in, or fixed on, any cosmetically or acceptable vector. The vector may be a particle with a range of sizes between 10 nm and 100 microns, more specifically 20 microns and 20 nm, and more specifically 15 microns and 100 nm. Particles may be composed of one or several excipients, including excipients generally regarded as safe (GRAS) by the FDA; phospholipids; saturated and unsaturated fatty acids and esters; PEGs of sizes from 100 g/mol to 20,000 g/mol, more specifically PEGs of 200 to 1200 g/mol, and more specifically PEGS of 400 to 600 g/mol; natural and unnatural polyamino acids having hydrophilic, hydrophobic, or chemically modified residues or some combination of these; synthetic and semisynthetic polymers such as HPMA, poly-lactic acid, and poly-esters; cyclodextrins; alcohols having one to twenty carbons; quaternary amines; lipids; fats; hydrophilic polymers; hydrophobic polymers; hydrogels; proteins; and any combination of the above or derivatives of the above.

It is of course obvious that the peptide according to the technology can be used alone or in association with at least one other active agent, in or for the preparation of a pharmaceutical composition.

In one embodiment, a particle having the peptide can be a certain size or within a certain size distribution. The size and zeta potential can be important as they are factors that impact the absorption of the formulation and peptide.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A method of treating or inhibiting a kidney disorder comprising:
providing an ASARM peptide; and
administering a therapeutically effective amount of the ASARM peptide to a subject to provide a treatment for the kidney disorder in the subject and/or inhibit development of the kidney disorder in the subject.

2. The method of claim 1, wherein the kidney disorder is selected from the group consisting of chronic kidney disease mineral bone disorder (CKD-MBD), calciphylaxis, nephrogenic systemic fibrosis (NSF), end stage renal disease, and combinations thereof.

3. The method of claim 1, further comprising inhibiting vascular calcification (VC), hard tissue calcification, non-vascular soft tissue calcification, mineralization, atherosclerotic plaque formation or combinations thereof in the subject with the ASARM peptide.

4. The method of claim 1, further comprising treating or inhibiting a metabolism abnormality of at least one of calcium, phosphorus, PTH, or vitamin D metabolism with the ASARM peptide.

5. The method of claim 1, further comprising treating or inhibiting an abnormality in at least one of bone turnover, bone mineralization, bone volume, bone growth, or bone strength with the ASARM.

6. The method of claim 1, further comprising treating or inhibiting hyperphosphatemia with the ASARM.

7. The method of claim 1, further comprising inhibiting nanocrystal or calciprotein particle formation with the ASARM by binding thereto.

8. The method of claim 1, further comprising inhibiting vascular smooth muscle cells (VSMC) from transitioning to an osteoprogenitor phenotype cell.

9. The method of claim 2, wherein the kidney disorder is CKD-MBD.

10. The method of claim 2, wherein the kidney disorder is calciphylaxis, which is inhibited from formation of lesions.

11. The method of claim 2, wherein the kidney disorder is NSF, and optionally the subject is administered the ASARM after receiving gadolinium binding contrast agents (GBCAs).

12. The method of claim 1, wherein the ASARM is infused into the subject.

13. A method of inhibiting a mineralization in a subject comprising:
providing an ASARM peptide; and
administering a therapeutically effective amount of the ASARM peptide to a subject to provide a treatment for inhibit mineralization in the subject.

14. The method of claim 13, wherein the ASARM inhibits mineralization of a soft tissue.

15. The method of claim 14, wherein the soft tissue is selected from brain, heart, renal, liver, muscle, dermal, vessel, tubule, or combinations thereof.

16. The method of claim 15, wherein the inhibited mineralization is inhibited calcification.

17. The method of claim 13, wherein the ASARM binds with mineral crystals.

18. A method of treating hyperphosphatemia comprising:
providing an ASARM peptide; and
administering a therapeutically effective amount of the ASARM peptide to a subject to provide a treatment for hyperphosphatemia.

19. The method of claim 18, wherein the ASARM reduces serum phosphate levels in the subject.

20. The method of claim 18, wherein the ASARM inhibits uptake of phosphate from by the digestive tract or kidney of the subject.

21. A method of treating or inhibiting an atherosclerotic disease, the method comprising:
providing an ASARM peptide; and
administering a therapeutically effective amount of the ASARM peptide to a subject to provide a treatment for the atherosclerotic disease in the subject and/or inhibit development of the atherosclerotic disease in the subject.

22. The method of claim 21, wherein the ASARM inhibits mineral plaque accretion and vascular occlusion.
